(19) United States
(12) Patent Application Publication
(10) Pub. No.: US 2013/0309295 A1
(43) Pub. Date: Nov. 21, 2013

(54) BIOSYNTHETIC FUNCTIONAL CELLULOSE (BC) FIBERS AS SURGICAL SUTURES AND REINFORCEMENT OF IMPLANTS AND GROWING TISSUE

(71) Applicant: Paul Gatenholm, Riner, VA (US)

(72) Inventor: Paul Gatenholm, Riner, VA (US)

(21) Appl. No.: 13/983,709

(22) PCT Filed: Feb. 6, 2012
(86) PCT No.: PCT/US2012/023963

§ 371 Date: Aug. 5, 2013

Related U.S. Application Data
(60) Provisional application No. 61/439,636, filed on Feb. 4, 2011.
(60) Provisional application No. 61/552,376, filed on Oct. 27, 2011.

(51) Int. Cl. C12P 19/04; A61L 27/20; A61L 17/10

(52) U.S. Cl. CPC C12P 19/04; A61L 17/10; A61L 27/20
USPC 424/444; 435/101; 536/56; 424/93.1

(57) ABSTRACT

Embodiments of the invention are based on the fermentation of bacteria to produce nano-cellulose in oxygen permeable tubular bioreactors. The resulting hydrogel non-hollow fiber can be stretched and dewatered to form strong, stiff yet flexible fiber. The fiber can be dehydrated by freeze drying or solvent exchange to form macroporous material and then optionally soaked with a solution of growth factors, anti-inflammatory drugs, and/or anitibacterial agents to provide a slow release drug delivery device in fiber form. The surface of the fiber is composed of nano-structured cellulose which promotes cell migration, tissue integration, and the healing process. BC fibers are not degraded in the human body and thus are well suited as reinforcement of implants and growing tissue. Uses for the BC fibers include surgical sutures, and reinforcing and promoting regeneration of damaged tissue or implants.

### CROSS-REFERENCE TO RELATED APPLICATIONS

The present application claims priority to and the benefit of the filing date of U.S. Provisional Patent Application No. 61/439,636 (filed Feb. 4, 2011) and No. 61/552,376 (filed Oct. 27, 2011), the disclosures of which are hereby incorporated by reference herein in their entireties.

### BACKGROUND OF THE INVENTION

1. Field of the Invention

The present invention relates to surgical materials, biomedical devices, tissue engineering, regenerative medicine, and health care products. More particularly, embodiments of the present invention relate to compositions of matter, and systems and methods for producing nano-cellulose biomaterials in the form of fibers produced by bacteria.

2. Discussion of Related Art

Surgical sutures are medical devices which are used to hold body tissues together after an injury or surgery. A number of different shapes, sizes, and materials have been used as sutures through history. In early days, plant materials such as flax, hemp and cotton (cellulose) or animal material such as hair, tendons, arteries, muscle strips and nerves, silk, catgut (collagen) were used. Today there are many absorbable synthetic polymeric sutures available on the market. In many applications non-absorbable sutures are however preferred.

In particular, however, there is a need for new materials which can promote the healing process and stimulate tissue regeneration. Strong biocompatible fibers are also of interest to use for reinforcement of tissue and scaffolds to guide the cells for repair and regeneration of organs such as tendons, ligaments, meniscus and guiding of neural cells.

Biosynthetic nano-cellulose, a natural polysaccharide, is an attractive biomaterial because of its good mechanical properties, hydroexpansivity, biocompatibility and its stability within a wide range of temperatures and pH levels. Cellulose (β-1→4-glucan) is the most abundant polymer of natural origin. In addition to being biosynthesized in vast amounts as structural material in the walls of plants, cellulose is also produced as an exopolysaccharide, i.e. bacterial cellulose (BC), by Gluconacetobacter xylinus.

Biomaterial applications require pure material and often an introduction of functional groups to stimulate the tissue regeneration process. BC has additional advantages as a biomaterial as compared to plant-derived cellulose. Apart from good mechanical strength, high water holding capacity, high purity and accessibility to non-aggregated micro fibrils, BC can be moulded into desirable shapes for a given application, allowing one to produce a three dimensional network of micro fibrils. Yet another interesting characteristic of BC microfibrils is the similarity in dimension with collagen micro fibrils, a common polymer in many tissues and a major constituent of bone.

Various attempts at preparing viable implantable BC type material have been made. For example, US Published Patent Application No. 20070128243 entitled “Implantable Microbial Cellulose Materials for Various Medical Applications,” which is incorporated by reference herein in its entirety, describes a method for preparing an implantable device for medical and surgical applications comprising: incorporating a material comprising microbial cellulose into an implantable device for repair or replacement of soft tissue. Also provided is an implantable composition comprising microbial cellulose, such as a tissue scaffold.

BC materials have also been used for hard tissue type regeneration, such as is described in US Published Patent Application No. 20070286884 entitled “Implantable Microbial Cellulose Materials for Hard Tissue Repair and Regeneration,” which is incorporated by reference herein in its entirety, and which describes an implantable composition comprising microbial cellulose and an agent for promoting hard tissue growth (such as a protein, a growth factor, or a drug).

Even further, it is known to use BC materials for reconstructive surgery, such as is provided by EP 2371401 entitled “A Method of Production of a Cartilage-Like Biomaterial Designed for Reconstructive Surgery,” which is incorporated by reference herein in its entirety, and which describes a method of preparing a cartilage-like biomaterial for reconstructive surgery implants by culturing microbial cellulose in a flat bioreactor or inside polyethylene tubes (a stationary culture of bacterium Gluconacetobacter xylinus), then purifying, rinsing, and modeling the material into a desired shape.

Manufacturing techniques for BC materials have also been refined. For example, it is known that BC hydrogel materials can be dried by methods such as critical point drying, freeze-drying, dewatering by organic solvents such as ethanol or acetone, air drying under normal or higher pressure, as well as hot-press drying. By drying under pressure, very flat foils (loss of 99% water) of high density and high mechanical stability can be obtained. In such materials, the nanofibril network collapses, resulting in a dense physical cross-linking of the cellulose chains (“hornification”). The material however absorbs a small amount of water and is thus not attractive as biomaterial.

Other preparation techniques are described for example in US Published Patent Application No. 20050042263 entitled “Dura Substitute and a Process for Producing the Same,” which is incorporated by reference herein in its entirety. This reference provides a method of producing a dural substitute comprising: producing a polysaccharide sheet; removing contaminants in said sheet; and dehydrating said sheet. Additionally, U.S. Pat. No. 6,599,518 entitled “Solvent Dehydrated Microbially-Derived Cellulose for in vivo Implantation,” which is incorporated by reference herein in its entirety, describes a method of making an implantable material for medical and surgical applications by treating a microbially-derived cellulose to render it non-pyrogenic, then dehydrating it chemically using methanol, ethanol, propanol, isopropanol, or acetone.

It is further known to construct BC materials into particular desired shapes during the growth process of the BC materials. For example in JP 3 272 772 A2 and EP 396 344 A2, which are hereby incorporated by reference herein in their entireties, it is described to use shaped bio-material as micro-luminal blood vessel substitutes, whereby the vessel prosthesis is cultivated on a hollow support which is permeable to oxygen (for example cellophane, Teflon, silicon, ceramic material, non-woven texture, fibers). The described process for producing the hollow microbial cellulose comprises the culturing of a cellulose synthesizing microorganism on the inner and/or outer surface of a hollow support permeable to oxygen, said support being made of cellophane, Teflon, silicon, ceramic material, or of a non-woven and woven material, respectively. Said hollow support permeable to oxygen is inserted into a culture solution. A cellulose synthesizing microorganism and a culture medium are added to the inner side and/or to the outer side of the hollow support. The culturing takes place under addition of an oxygenous gas (or liquid) also to said inner side and/or to the outer side of the hollow support. A gelatinous cellulose of a thickness of 0.01 to 20 mm forms on the surface of the support.

Another process for producing hollow microbial cellulose is described in EP 396 344 A2, also incorporated by reference herein in its entirety, which provides a method of manufacturing by way of two glass tubes of different diameter. The glass tubes are inserted into one another and culturing of the microorganisms is carried out in the space between the two tube walls within 30 days. The result is microbial cellulose of a hollow cylindrical shape and evaluated for its blood compatibility, antithrombogenic property by a blood vessel substitute test in a dog. Likewise, WO 01/610 26 A1 and (Klemm et al. Prog. Polymer Sci. 26 (2001) 1561-1603) describe a method for producing shaped biomaterial by means of culturing cellulose producing bacteria in a cylindrical glass matrix, in particular for microsurgical applications as blood vessel substitutes of 1-3 mm diameter and smaller. These references are also incorporated by reference herein in their entireties.

Additionally, US Published Patent Application No. 20100042197 entitled “Preparation of Hollow Cellulose Vessels,” which is incorporated by reference herein in its entirety, describes hollow cellulose vessels, tubes, artificial blood vessels, and patches prepared by culturing cellulose-producing microorganisms on the outer surface of a hollow carrier, and providing an oxygen containing gas on the inner side of the hollow carrier, wherein the oxygen containing gas has an oxygen level higher than atmospheric oxygen.

International Patent Application Publication No. WO 89/12107 describes various methods for producing microbial cellulose at a gas/liquid interface, where the yield of cellulose can be improved by increasing the concentration of oxygen available to the bacteria by bubbling, agitation or increasing the pressure or concentration of oxygen in the ambient gas environment.

Oxygen is reported to be a limiting factor for the yield of microbial cellulose produced (Schramm & Hestrin J. Gen. Microbiol. 11 (1954) 123-129. On the other hand Watanabe et al. (Biosci. Biotechnol. Biochem. 59 (1995) 65-68) reported that a higher oxygen tension in the gaseous phase than atmospheric air inhibits BC production.

U.S. Pat. No. 6,017,740 and corresponding EP 0792935 describe a process for the production of bacterial cellulose in an aerated and agitated fermentation tank and increased oxygen pressure and content are used to increase the yield of microbial cellulose.

US Published Patent Application No. 20100297239 entitled “Osseointegrative Meniscus and Cartilage Implants Based on Beta-Glucan Nanocomposites,” which is incorporated by reference herein in its entirety, describes medical implants to treat meniscus and cartilage damage produced by a method of culturing a microorganism on a solid substrate by providing to the microorganism a first level of oxygen to cause the microorganism to produce a first type of glucan units resulting in deposition of cellulosic fibrils on the solid substrate, and providing to the microorganism a second level of oxygen to cause the microorganism to produce a second type of glucan units resulting in production of a hydrogel.

A major limitation of cellulose fibers derived from plants (cotton, flax, linen) for use in surgical applications is lack of control of size and shape, poor mechanical properties and in some extent foreign body reaction. In contrast to plant derived cellulose one can control the size and shape of cellulose nanofibrils produced by bacteria such as Gluconacetobacter xylinus. The growing glucan chains aggregate and are exported through catalytic sites that are linearly arranged on each bacteria cell. Bacteria assemble glucan chains into microfibrils and subsequently into a ribbon configuration. In the normal static conditions bacteria will form a pellicle (flake) at the surface of the culture medium. Shaped cellulose tubes (hollow) with limited thickness has been produced in the method using tubular bioreactor as described in WO2001061026. The oxygen delivery through the silicon support has been explored for manufacturing of tubes for applications as vascular grafts as described in EP2079845 and WO2008040729 A2.

Notwithstanding various attempts in the art to design implantable biomaterials from BC, there remains the need to be able to grow BC materials especially in the form of sutures having a desired form and morphology, as well as strength, elasticity, and high porosity.

### SUMMARY OF THE INVENTION

To this end, it has been discovered that it is advantageous to cultivate bacteria inside permeable tubes. Instead of producing hollow tubes by growing BC on the outside of the substrate, a compact gel (non-hollow) in the form of a substantially cylindrical fiber can be grown. Such gel when stretched and dewatered under well defined conditions forms robust fiber with controlled size. Optionally, the fiber-gel can be dehydrated before stretching by freeze drying or solvent exchange. The resultant macroporous fiber can be resoaked in solution or suspension or dispersion of growth factors, drugs, electroconductive polymer or antimicrobial agents. When the “loaded” fiber is stretched it can incorporate the active agent and then release from fiber upon use. In embodiments, the fiber has nanostructured surface which is promoting cell and tissue interactions.

More particularly, a biofabrication process has been developed in which bacteria suspension in media is injected into gas permeable hollow fiber and then cultivated for 2-5 days. Bacteria Gluconacetobacter Xylinus produce pre-oriented nano-cellulose network which is a compact hydrogel in the form of fiber. The length of the fiber can be up controlled and adjusted for example for 50 cm which corresponds to length of single suture package. The diameter of the hydrogel can vary between 1 and 3 mm. After cultivation process the hydrogel fiber is washed to remove bacterial cells. Wet hydrogel fiber is then carefully stretched and dewatered which result in highly oriented fiber structure with good mechanical performance and silk-like feeling, perfect material for surgical suture applications. Wet hydrogel can be dehydrated by freeze drying or solvent exchange. Resulting macroporous fiber can be soaked in solution or suspension or dispersion of active agent. After “loading” the hydrogel fiber can be stretched and form oriented cellulose fiber containing active agents. These agents can slowly release from fiber improving cell attachment, cell differentiation, guiding the cells, and improving the healing process. It is expected that the BC fibers will be biocompatible, integrated in the tissue, promote healing process by cell attachment and remain its mechanical properties with time. They can be used for many biomedical applications such as sutures, reinforcement of tissue, repair and regeneration of tendon, ligaments, menisci and guide of neural cells.

In particular, embodiment of the present invention provide a process for producing biosynthetic cellulose (BC) fibers comprising: injecting a suspension of cellulose producing bacteria in medium for growing BC hydrogel into oxygen permeable tubing with a diameter of less than 3 mm; growing BC hydrogel in the tubing to a desired length and diameter and having a desired morphology, porosity, and mechanical properties; and stretching and drying the BC hydrogel to align cellulose nanofibrils.

Such processes can further comprise combining the BC hydrogel with growth factor or anti-inflammatory drugs before drying. Likewise, such processes can further comprise surface modifying the BC hydrogel with antimicrobial agents to produce antimicrobial BC fibers.

Embodiments include BC fibers formed from the processes described in this specification, as well as surgical sutures formed from BC fibers produced according to this specification, especially those that can be used as surgical sutures.

The present invention also provides in embodiments, a drug delivery device comprising BC fiber produced according to methods described herein. BC fiber(s) produced according to methods described in this specification can be used for drug delivery.

A method of reinforcing a medical implant or biomedical device is also encompassed by embodiments of the invention and can comprise using BC fibers produced according to methods described in this specification to secure the implant or device in a body.

BC fibers produced according to this specification can be useful in tissue repair applications. Methods of the invention include seeding the BC fibers with cells, if desired, to encourage and/or facilitate tissue growth into, on, and/or through the engineered BC fibers.

For example, a method of tissue regeneration according to embodiments of the invention can comprise seeding BC fibers produced according to methods described in this specification with cells and contacting the fibers with damaged tissue. Contact between the tissue and fibers can be achieved in any number of ways according to usual surgical techniques, including weaving the BC sutures into and/or through the tissue or organ, placing the BC fibers on the tissue or organ, and/or securing the BC fibers to the tissue to ensure contact between the fiber and the tissue. The BC fibers can be used with healthy tissue to encourage additional new growth in certain situations or can be used to repair damaged tissue. Along these lines, BC fibers produced according to methods described herein for use in tissue regeneration are also included as an aspect of embodiments of the present invention.

### BRIEF DESCRIPTION OF THE DRAWINGS

The accompanying drawings illustrate certain aspects of some of the embodiments of the present invention, and should not be used to limit or define the invention. Together with the written description the drawings serve to explain certain principles of the invention.

FIG. 1 is a schematic diagram showing a representative experimental set up in which bacteria suspension in medium can be injected into oxygen permeable silicon tubing, where a set of multiple tubings can be connected into a cartridge (hollow fiber cartridge) and several cartridges can be connected in the bioreactor production system.

FIG. 2 is a photograph of a representative BC hydrogel fiber in suture form having a diameter of about 200 micron capable of stretching under controlled conditions.

FIG. 3 is a graph showing the stress-strain behavior of BC hydrogel fiber under stretching and dewatering which converts hydrogel into BC fiber, where specimen 1 is wet hydrogel and specimen 2 is partially dewatered material.

FIG. 4 is a micrograph showing a BC suture with diameter of about 200 micron prepared by cultivation in 1.4 mm silicone tubing for 4 days, purified and stretched.

FIGS. 5A-B are Scanning Electron Microscopy (SEM) images of typical distribution of nano-cellulose fibrils in BC hydrogel (left—the nanofibrils are randomly distributed) and in BC fiber (right—the nanofibrils are assembled and oriented in fiber direction).

FIG. 6 is an SEM image illustrating the fracture surface of BC suture with diameter of 200 micron after tensile test, which shows orientation of nanofibrils and fracture of single fibrils upon tensile loading.

FIGS. 7A-B are SEM images of circumferential collagen fibers in human meniscus (left) and the surface of BC fiber with diameter of 200 micron (right), which shows BC fiber has a hierarchical organization similar to that of native tissue.

FIGS. 8A-B are SEM images of oriented nanocellulose fibrils in functional BC fiber (left), and human mesenchymal stem cells grown on the surface of BC fiber containing sodium hyaluronate (right).

### DETAILED DESCRIPTION OF VARIOUS EMBODIMENTS OF THE INVENTION

Reference will now be made in detail to various exemplary embodiments of the invention. It is to be understood that the following discussion of exemplary embodiments is not intended as a limitation on the invention. Rather, the following discussion is provided to give the reader a more detailed understanding of certain aspects and features of the invention.

Biosynthetic nano-cellulose, such as that produced by the bacteria Gluconacetobacter xylinus, or other appropriate bacteria, is an emerging biomaterial with great potential as a biological implant, wound and/or burn dressing material, and as scaffolds for tissue regeneration. It has excellent mechanical properties and does not elicit foreign body reactions. It is also attractive for cell immobilization and cell support.

Biosynthetic nano-cellulose is a biosynthetic material that is not recognized as foreign material by the body; in other words, it mimics the absorbable suture materials favored by surgeons. Among benefits exhibited by such nano-cellulose include that the material exhibits the ease of use (easy to use in closing the wound and in forming knots) of monofilament materials that surgeons require; provides the knot security required by surgeons; does not cause a chronic inflammatory response in the body; in other words, it mimics an absorbable suture material; and provides the tensile strength required to close the wound and maintains sufficient strength for the wound to heal properly.

In addition, the nano structure of BC Suture tends to promote cell migration and interaction, which can be critical for wound healing and tissue regeneration; leads to minimal scarring due to the high biocompatibility of BC suture; potential modifications of BC sutures that take advantage of its nano structure and would allow slow release in the wound region; incorporation of select growth factors; incorporation of anti-inflammatory drugs; and incorporation of antimicrobial agents can also be used.

A novel method to grow BC hydrogel fiber with controlled length and diameter has been invented. Optionally, such hydrogel fiber can be loaded with growth factors, anti-inflammatory drugs and antimicrobial agents and stretched and dehydrated.

An exemplary process starts with injection of bacteria suspended in suitable medium into PDMS (polydimethylsiloxane) tubing with any length but thin wall (less than 500 micron) to allow oxygen to permeate and inner diameter less than 3 mm (to allow nano-cellulose hydrogel to fill the whole volume of the tubing. The wall of the tubing can be for example from 10-500 micron, such as from 50-400 micron, such as from 100-300 micron, such as from 75-250 micron, and including from 200-350 micron. Further, the diameter of the tubing can range from about 0.2 mm to about 5 mm, but is preferred to be less than about 3 mm in diameter. For drug delivery applications, larger diameter BC fibers may be desired, yet for sutures smaller diameters may be desired. Preferred diameters of the tubing can range from 0.5 mm to 2 mm, such as from about 1 mm to 2.5 mm, or any size in between. Even further, because cylindrical tubing is typically used due to availability, the fibers will be generally cylindrical in shape. Any shape tubing can be used, however, including square, rectangular, or triangular, to name a few.

The incubation process generally takes between 3 and 5 days, but the bacteria can be incubated for any desired amount of time from 1-30 days to achieve a particular desired result. Optionally, the environment around the silicone tubing can be enriched in oxygen in order to increase production of cellulose in the tubing but it is also preferred to add an element of humidity to the environment to avoid any drying of solution in the tubing.

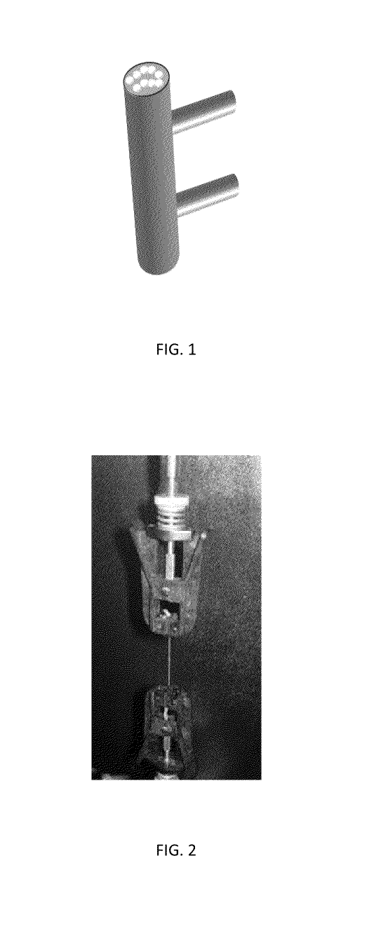

Several PDMS tubings (for example 50) can be packed into a plastic or glass or metal cartridge, similar to one used as hollow fiber filtration unit. Any number of tubings from 1-500 indeed can be used. FIG. 1 shows a typical cartridge which can be used to perform the work described above. After 3-5 days, the tubing is filled with gel like material which is nano-cellulose. It is of outermost importance that the hydrogel fiber is not hollow. It has to be compact. Growing the hydrogel on the outer surface of the tubing to result in hollow tubular shaped fibers results in an overall non-compact fiber.

The hydrogel fiber is washed with alkali to remove bacteria and with water and then stretched under well defined conditions. FIG. 2 shows how the fiber hydrogel is stretched in a tensile testing machine under well controlled conditions of load and extension rate. The hydrogel dries upon stretching and the cellulose nanofibrils which are typically randomly oriented are as a result aligned.

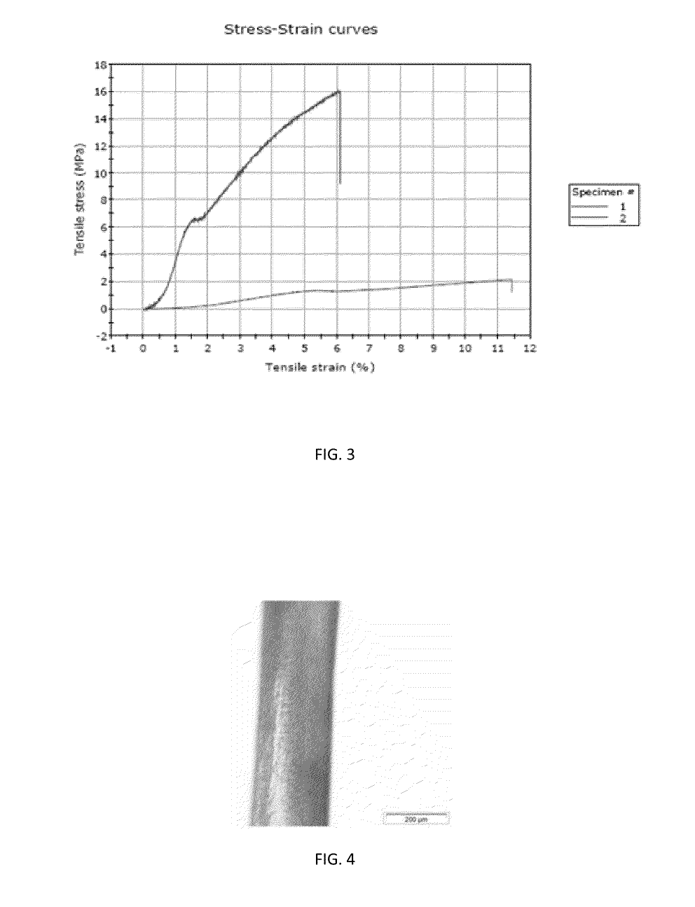

FIG. 3 shows stress strain behavior of wet hydrogel fiber (lower curve) and pre stretched fiber (upper curve). After stretching the compact BC fiber is obtained. The BC fiber shown in the FIG. 4 has diameter of about 200 micron, which was grown in the 1.4 mm inner diameter PDMS tubing.

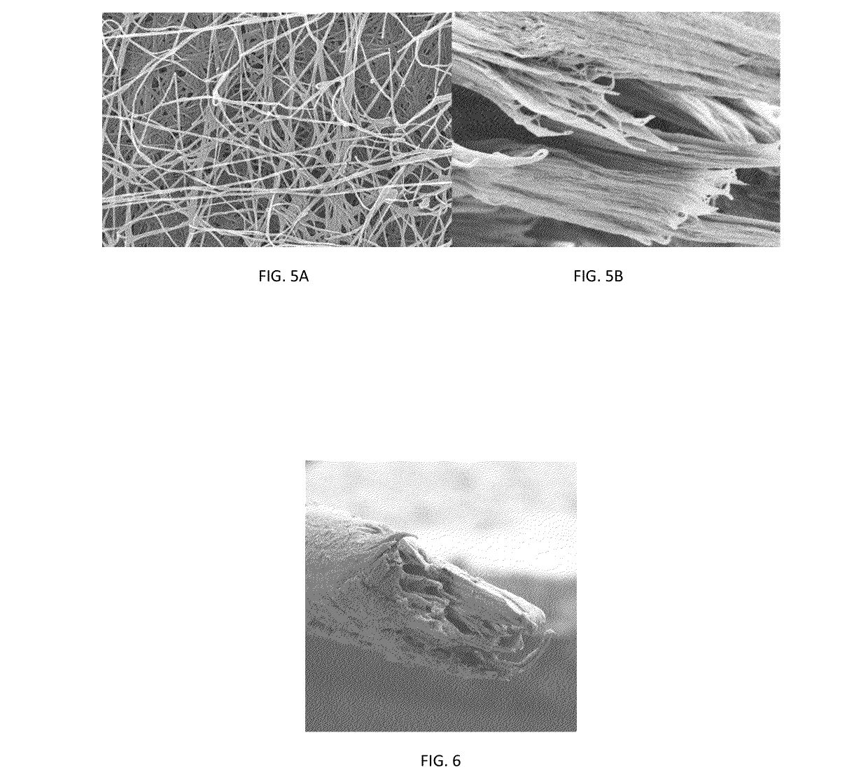

FIG. 5 shows how the morphology of nano-cellulose hydrogel has been changed during stretching and dewatering process. The left SEM image on FIG. 5A shows random distribution of nano-cellulose network and the right SEM image on FIG. 5B shows oriented and dense nano-cellulose network.

The BC fibers have been evaluated for applications as surgical sutures. The most important property of surgical sutures are the mechanical properties. Mechanical properties were evaluated in stress-strain mode and the strength was 95 MPa and stiffness was 4.4 Gpa and elongation to break was 3.4%. FIG. 6 shows SEM image of fracture surface of BC fiber. It is clearly shown the alignment and fracture of individual fibrils.

Any combination of mechanical properties can be achieved according to the processes of the present invention. For example, BC fibers can be grown comprising a strength in the range of 50-300 MPa, such as from 60-200 MPa, such as from 75-150 MPa, such as from about 80-120 MPa, and including from about 90-115 MPa. Alternatively, or in addition, the BC fibers can have a stiffness of about 1-10 GPa, and more preferably from about 2-8 GPa, such as from about 3-7 GPa, or from about 4-6 GPa, even including from about 4.2 to 5.2 GPa, or from about 3.8 to 4.6 GPa, or even from about 3.6 to 4.6 GPa and so on. Elongation to break is another favorable characteristic of sutures according to embodiments of the invention. The BC fibers can alternatively or in addition to other mechanical properties described in this specification comprise an elongation to break of up to approximately 20%. Preferred BC fibers can have an elongation to break ranging from about 1-15%, or from about 2-12%, or from about 3-10%, or from about 4-6%, or from about 2.5-4.5%, such as from about 2.8-3.6%, or from about 2.9-3.5%, or from about 3.1-3.3%, and so on. The mechanical properties of the BC fibers can be purposefully selected and obtained using the growth processes of the present invention.

BC fibers are robust and are very attractive as surgical sutures. The process of production allows incorporation of growth factors or anti-inflammatory drugs in gel phase. Furthermore the surface can be modified with silver nanoparticles to provide antimicrobial properties, if desired for certain applications.

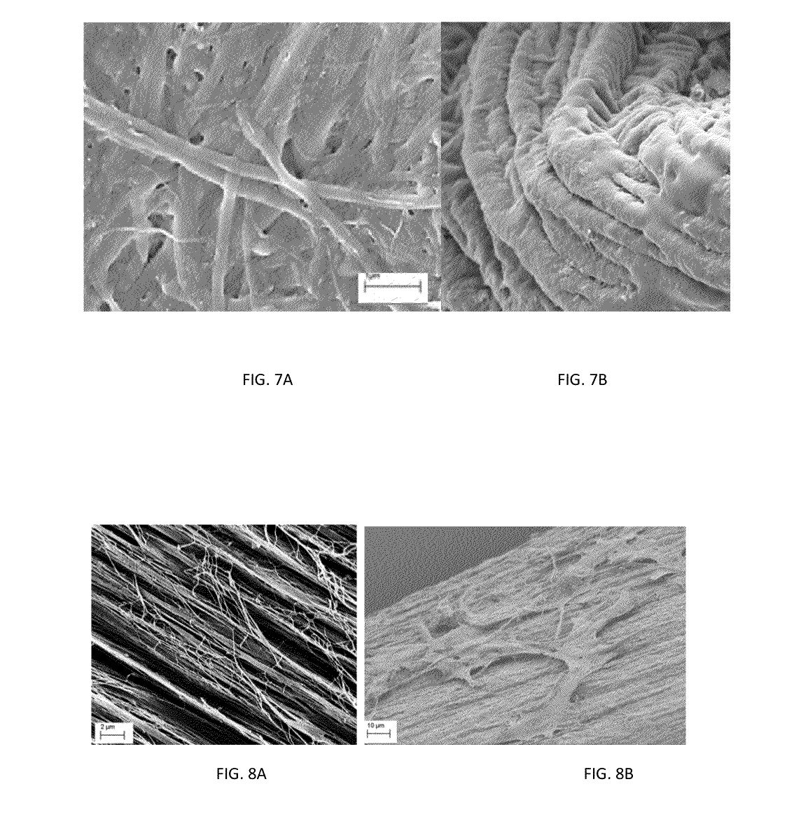

There are however other applications of these novel BC fibers. The surface of the fibers is composed of nano-structured cellulose fibrils. Such BC fibers are very similar to collagen fibers which are load bearing in tissues such as tendons, ligaments or circumferential fibers in meniscus. FIG. 7A (left) shows collagen circumferential fibers in human meniscus.

FIG. 7B (right) shows the surface of BC fibers. BC fibers can be used as replacement of ligaments, tendons or reinforcement of meniscus implant. They can also be used as reinforcement of any growing tissue. FIG. 8A (left) shows typical strongly oriented assembled nanocellulose fibrils in the functional BC fiber which was “loaded” with sodium hyaluronate which can be slowly released from the functional BC fiber promoting cell attachment. FIG. 8B (right) shows strongly attached to the BC fiber surface human mesenchymal stem cells. Such good adhesion is required for cell differentiation and support of cell growth in new tissue and organs such as tendons, ligaments, nerves, muscles etc.

The process described is composed of one or more of several elements: (1) Cells which are capable of synthesizing one or more extracellular biopolymers of interest; (2) Media in which the cells can be suitably maintained under conditions conducive to the bioproduction of the one or more extracellular polymers of interest; (3) Tubular oxygen permeable bioreactor; (4) A device to stretch hydrogel fibers with simultaneous dewatering.

The system described above can additionally be equipped with customizable form to produce predetermined 3D shape and connected with device which adds porogens in controlled manner. The components are discussed in detail below:

CELLS: The extracellular biopolymers that are synthesized or produced by the cells that are employed in the invention include cellulose. The cellulose producing bacteria may be Gluconacetobacter, Agrobacterium, Rhizobium, Pseudomonas or Alcaligenes most preferably species of Acetobacter xylinum or Acetobacter pasteurianus. The most preferred strain is Gluconacetobacter xylinus subsp.sucrofermentas BPR2001, trade number 700178™, from the ATCC. In addition, the cells may be genetically engineered to control other useful properties, including but not limited to their charge; the ability to produce a biopolymer if they do not naturally do so; the ability to produce more than one bioplymer, e.g. to produce one or more biopolymers in addition to those that they naturally produce. In addition, any of the strains of bacteria noted in any of the references incorporated by reference in this specification can be used in embodiments of the invention. Likewise, suitable media for growing BC in such bacteria can be obtained from any reference cited and incorporated by reference herein.

MEDIUM: The medium in which the cells are maintained during biopolymer production may be any of many suitable types. The medium is generally liquid, and of a viscosity that allows the cells to move or be moved through the medium in response to directional prompting by application of an electromagnetic field. The viscosity of the medium may be altered to produce desired speeds of movement or patterns of distribution of the cells. Further, in some embodiments, the medium may be a gel. In this embodiment, the movement of the cells may be somewhat curtailed, but the imposed electromagnetic field is still capable of eliciting movement such as orientation of the cells, spinning in place, etc. Deposition of polymers in gels may produce more tightly packed polymer formations.

Those of skill in the art are generally familiar with the culture of cells in liquid suspension. Such cultures are usually aqueous, and contain various nutrients and supplements that permit growth and/or maintenance and metabolic activity of the cells, and are suitably oxygenated or not, depending on the requirements of the cells. The nutritive components of the medium may be used by the cell for general metabolic and catabolic activities, as well as to build the biopolymer(s) of interest. Further, the medium may be supplemented in particular to support biopolymer synthesis (e.g. by providing an abundant source of e.g. monomeric polymer building blocks, or to bias the cellular metabolism in favor of biopolymer synthesis, etc.).

Examples of suitable media for growing bacteria include but are not limited to: Schramm-Hestrin-medium which contains, per liter distilled water, 20 g of glucose, 5 g of bactopeptone, 5 g of yeast extract, 3.4 g of disodium-hydrogenphosphate dehydrate and 1.15 g of citric acid monohydrate and which exhibits a pH value between 6.0 and 6.3; 0.3 wt % green tea powder and 5 wt % sucrose with pH adjusted to 4.5 with acetic acid; Medium composed of (fructose [4% w/v], yeast extract [0.5% w/v], (NH4)2SO4 [0.33% w/v], KH2PO4 [0.1% w/v], MgSO4.7H2O [0.025% w/v], corn steep liquor [2% v/v], trace metal solution [1% v/v, (30 mg EDTA, 14.7 mg CaCl2.2H2O, 3.6 mg FeSO4.7H2O, 2.42 mg Na2MoO4.2H2O, 1.73 mg ZnSO4.7H2O, 1.39 mg MnSO4.5H2O and 0.05 mg CuSO4.5H2O in 1 liter distilled water)] and vitamin solution [1% v/v (2 mg inositol, 0.4 mg pyridoxine HCl, 0.4 mg niacin, 0.4 mg thiamine HCl, 0.2 mg para-aminobenzoic acid, 0.2 mg D-panthothenic acid calcium, 0.2 mg riboflavin, 0.0002 mg folic acid and 0.0002 mg D-biotin in 1 liter distilled water)]). Any medium comprised of sugar source, nitrogen source and vitamins can be successful used. Bacteria grow even in apple or pineapple juice, coconut milk, beer waste, or wine.

The media may be altered to include ions such that ions are deposited onto the biopolymer. This can include but are not limited to: Schramm-Hestrin-medium with 1, 5, or 10% PBS (Phosphate Buffered Saline), Schramm-Hestrin-medium with 1%, 5%, or 10% 0.1 molar calcium chloride, or any suitable culture media with an increased concentration of one or more ions. Ions may include but are not limited to potassium, calcium, phosphate, or sodium.

Any combination of bacteria, media, and growing conditions can be used to prepare the controlled morphology, controlled mechanical properties, cylindrical BC fibers according to the invention. Although specific examples are provided below, these examples can be modified in light of any of the references cited and incorporated by reference in this specification to obtain a specific desired result.

### EXAMPLES

### Example 1

### Production of BC Hydrogel Fiber

A representative production system for the controlled growth of BC hydrogel fiber is shown schematically in FIG. 1. The system comprises a cartridge containing sterile silicone tubing. Silicone tubing (PDMS) from Lebo Sweden, with diameter of 1.4 mm and a Shore A hardness of 50 were used in this example. Any type of silicone rubber tubing can be used. Characteristics of the tubing can include that it is generally non-reactive, stable, and resistant to extreme environments and temperatures from −120° C. to +300° C. Polydimethylsiloxane (PDMS) is a preferred type of tubing that can be used according to methods of the invention. Mechanical properties of the rubber tubing can include: a Shore A Hardness in the range of about 3-90, such as from about 5-10, 20-80, or about 30-70, or about 40-60, or about 15-65, or about 25-45, or about 35-55, or any hardness value in this range; a tensile strength of the material of the rubber tubing can be in the range of about 5-20 N/mm², such as from 8-15 N/mm², or such as from 10-18 N/mm², or such as from 12-16 N/mm², and any tensile strength in this range; an elongation at break ranging from 100-1100%; a density of about 1.05-1.60 g/cm³; a tear strength (ASTM D 624) in the range of about 5-55 N/mm; and/or a rebound resilience ranging from about 30-70%.

A suspension of bacteria in culture media suitable for growing BC hydrogel was used. In particular, 10 ml of medium (composed of fructose [4% w/v], yeast extract [0.5% w/v], (NH4)2SO4 [0.33% w/v], KH2PO4 [0.1% w/v], MgSO4.7H2O [0.025% w/v], corn steep liquor [2% v/v], trace metal solution [1% v/v, (30 mg EDTA, 14.7 mg CaCl2.2H2O, 3.6 mg FeSO4.7H2O, 2.42 mg Na2MoO4.2H2O, 1.73 mg ZnSO4.7H2O, 1.39 mg MnSO4.5H2O and 0.05 mg CuSO4.5H2O in 1 liter distilled water)] and vitamin solution [1% v/v (2 mg inositol, 0.4 mg pyridoxine HCl, 0.4 mg niacin, 0.4 mg thiamine HCl, 0.2 mg para-aminobenzoic acid, 0.2 mg D-panthothenic acid calcium, 0.2 mg riboflavin, 0.0002 mg folic acid and 0.0002 mg D-biotin in 1 liter distilled water)]) was sterilized by microfiltration and placed in a sterile syringe. 0.5 ml of Gluconacetobacter xylinus subsp.sucrofermentas BPR2001, 700178™, from the ATCC bacteria suspension taken from preculture was added to 10 ml of medium.

The bacteria suspension was injected into the silicone fibers placed in the cartridge (FIG. 1). The cartridge was placed in an incubator holding 30 degree Celsius for 3 days. Any amount of incubation time can be used to achieve a desired growth. Preferably the time can be varied between 2 and 5 days. The most important factor is to fill the tubing by BC hydrogel. It is preferential to keep the temperature of medium at about 30 degrees C. The production of cellulose starts at the inner surface of the silicone tubing and proceed toward the middle of the tubing. BC layers form most readily at the intersection of the solid, liquid, air boundary. Avoidance of drying of the cellulose can be achieved by holding high moisture content around the silicone tubing in the cartridge. In some experiments oxygen gas can be introduced into the system (still keeping high moisture content) outside the silicone tubing to increase cellulose production. The lengths of tubing can be varied and adjusted to a desired need. In this example, 50 cm long tubing was used.

### Example 2

### Conversion of BC Hydrogel Into Robust BC Fiber

The BC fiber hydrogel produced in Example 1 was washed with 0.1 m alkali solution for 1 hour at 60 degrees Celsius and then washed several times with distilled water. The hydrogel fiber was then removed from the silicone tubing and stretched using a tensile testing machine (FIG. 2). Different strain rates were evaluated and it was found that 10% stretching imparted the BC fibers with optimal mechanical properties. It is important to keep BC hydrogel wet upon stretching since water acts as plasticizer and allows nano-cellulose to align and achieve orientation in the direction of stretching. In preferred embodiments the hydrogel is slowly dewatered upon stretching to yield robust BC fiber as shown in FIG. 4.

The BC fibers were evaluated for applications as surgical sutures. The most important property of surgical sutures are the mechanical properties. Mechanical properties were evaluated in stress-strain mode and the strength was 95 MPa and stiffness was 4.4 Gpa and elongation to break was 3.4%. FIG. 6 shows SEM image of fracture surface of BC fiber. It is clearly shown the alignment and fracture of individual fibrils.

### Example 3

### Use of BC Fibers as Reinforcement of BC Meniscus Implant

The BC fibers with a diameter of 200 microns produced in Example 1 and converted into robust fibers as described in Example 2 were sterilized in autoclave and evaluated as reinforcement of BC meniscus implant. The BC fibers were placed in meniscus bioreactor in circumferential direction and BC meniscus was grown in such bioreactor using 3D Bioprinting process. After 2-24 hours, when a confluent layer of BC was produced in the meniscus form, addition of a suitable medium was started at the rate which was matching BC production in the mold. After 5 days of such culture the robust BC meniscus in the shape of a patient's injured meniscus was produced. The BC meniscus was removed from the mold, washed in 0.1 m NaOH solution at 60 degree Celsius for 48 hours and then washed several days with DI water. After sterilization in an autoclave BC meniscus implant was ready for use as implant. It was found that the BC fibers were very well integrated into the BC meniscus structure and thus reinforcing the implant exactly the way as circumferential collagen fibers reinforce native meniscus.

### Example 4

### Preparation and Use of Functional Biofibers for Stem Cell Differentiation and Tissue Regeneration

The BC fiber hydrogel produced in Example 1 was washed with 0.1 m alkali solution for 1 hour at 60 degree Celsius and then washed several times with distilled water. The hydrogel fiber was then removed from the silicone tubing and dehydrated by freezing at −80 degrees Celsius and then freeze drying for 24 hours. Macroporous material with preoriented nanocellulose fibrils was obtained in such process. Macroporous material was soaked in 1% water solution of sodium hyaluronate. After soaking, the material was stretched in a tensile testing machine according to experimental procedure described in Example 2. The sodium hyaluronate solution can be replaced by anti-inflammatory drugs, growth factors, anti-microbial agents or conductive polymers. During the stretching process pre-oriented BC nanofibrils are agglomerated and assembled into fiber leaving the second phase of added component imbedded between the fibrils. This is beneficial to proceed in such way such that incorporation of one or more functional agent will not disturb fiber formation. The functional agent can slowly diffuse out from the fiber surface. In such a way the functional BC fiber act as slow release device. The BC fibers containing sodium hyaluronate were used as substrate for growth of human mesenchymal stem cells. Cells were strongly attracted to the functional BC fiber and were adhered strongly to the fiber surface. This shows that such functional fiber can be use to differentiate cells and support growth of tissue like ligament, tendon or nerves.

The present invention has been described with reference to particular embodiments having various features. It will be apparent to those skilled in the art that various modifications and variations can be made in the practice of the present invention without departing from the scope or spirit of the invention. Indeed, numerous references have been cited in this specification to provide background about the current state of the art. These references can be used by those skilled in the art to supplement this disclosure. To this extent, all of the references cited in this specification are hereby incorporated by reference herein in their entireties to form part of the disclosure of the preferred embodiments of the present invention. For example, any of the techniques described in those references can be applied to the processes described herein as part of the embodiments of the invention. In particular, for the constituents of suitable culture media for growing BC in bacteria, any culture media described in the references provided in the specification can provide specific examples applicable to preparation of the BC fibers disclosed herein or can provide guidance as to certain parameters that may be desired for a certain result. Likewise, for increasing and/or altering the growth rate of BC using oxygen, one skilled in the art can refer to techniques provided in the cited references for specific guidance. One skilled in the art will recognize that the features of embodiments of the invention may be used singularly or in any combination based on the requirements and specifications of a given application or design, and one or more elements, constituents, or process steps may be omitted, incorporated, or altered as desired. Other embodiments of the invention will be apparent to those skilled in the art from consideration of the specification and practice of the invention. It is intended that the specification and examples be considered as exemplary in nature and that variations that do not depart from the essence of the invention are intended to be within the scope of the invention.

The present invention is well adapted to attain the ends and advantages mentioned as well as those that are inherent therein. The particular embodiments disclosed above are illustrative only, as the present invention may be modified and practiced in different but equivalent manners apparent to those skilled in the art having the benefit of the teachings herein. It should be evident that the particular illustrative embodiments disclosed above may be altered or modified and all such variations are considered within the scope and spirit of the present invention. While compositions and methods are described in terms of “comprising,” “containing,” or “including” various components or steps, the compositions and methods can also “consist essentially of” or “consist of” the various components and steps. All numbers and ranges disclosed above may vary by some amount. Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the element that it introduces. If there is any conflict in the usages of a word or term in this specification and one or more patent or other documents that may be incorporated herein by reference, the definitions that are consistent with this specification should be adopted.

1. A process for producing biosynthetic cellulose (BC) fibers comprising:
injecting a suspension of cellulose producing bacteria in medium for growing BC hydrogel into oxygen permeable tubing with a diameter of less than 3 mm;
growing BC hydrogel in the tubing to a desired length and diameter and having a desired morphology, porosity, and mechanical properties; and
stretching and drying the BC hydrogel to align cellulose nanofibrils.

2. The process of claim 1 further comprising combining the BC hydrogel with growth factor or anti-inflammatory drugs before drying.

3. The process of claim 1 further comprising surface modifying the BC hydrogel with antimicrobial agents to produce antimicrobial BC fibers.

4. BC fibers formed from the process of claim 1.

5. Surgical sutures formed from the BC fibers produced in claim 1.

6. BC fiber produced according to claim 1 for use as surgical sutures.

7. A drug delivery device comprising BC fiber produced according to claim 3.

8. BC fiber produced according to claim 3 for use in drug delivery.

9. A method reinforcing a medical implant or biomedical device comprising using BC fibers produced according to claim 1 to secure the implant or device in a body.

10. BC fibers produced according to claim 1 for use in tissue repair.

11. The method of claim 9, further comprising seeding the BC fibers with cells.

12. A method of tissue regeneration comprising seeding BC fibers produced according to claim 1 with cells and contacting the fibers with damaged tissue.

13. The method of claim 12, wherein contacting comprises securing the BC fibers to the damaged tissue.

14. BC fibers produced according to claim 1 for use in tissue regeneration.
